(19) United States
(12) Patent Application Publication
(10) Pub. No.: US 2011/0287524 A1
(43) Pub. Date: Nov. 24, 2011

(54) METHODS AND SYSTEMS FOR PREDICTIVE DESIGN OF STRUCTURES BASED ON ORGANIC MODELS

(75) Inventor: Frederic Zenhausern, Fountain Hills, AZ (US)

(73) Assignee: DIOMICS CORPORATION, La Jolla, CA (US)

(21) Appl. No.: 13/097,030

(22) Filed: Apr. 28, 2011

Related U.S. Application Data
(60) Provisional application No. 61/343,366, filed on Apr. 28, 2010.

(51) Int. Cl. C12N 1/12; C08G 77/00; G06F 17/50

(52) U.S. Cl. 435/257.1; 703/1; 521/154

(57) ABSTRACT

A method and system to predict a design of an article of manufacture, which can include the steps of introducing a biological organism into a test environment to activate at least one molecular component of the biological organism representing at least one set of genetic characters of the biological organism; monitoring for change in at least one expression phenomenon pattern in response to the test environment; translating the monitored change in the at least one expression phenomenon pattern into a molecular data profile; interrogating the molecular data profile against a stored database to generate an information pattern of a phenotype correlated to the molecular data profile; and translating the information pattern of the phenotype into a correlated design component of the article of manufacture.

### CROSS-REFERENCE TO RELATED APPLICATION

This application claims priority to U.S. Patent Appl. No. 61/343,366, filed Apr. 28, 2010, which is incorporated herein by reference in its entirety.

### FIELD OF THE INVENTION

Methods and systems for predicative design of an article of manufacture are described herein and, in particular, methods and systems for predicative design of an article of manufacture based on organic models.

### BACKGROUND

Materials processing and development of new products can often require costly and time consuming prototype designs tested under a variety conditions using trial and error. Frequently, a design must be adjusted multiple times before an acceptable or adequate design is achieved. Even under these expensive and time consuming circumstances designs may still not be optimized.

One attempt in the art to reduce product design costs can include the use of a predictive design. Predictive design is a discipline that has traditionally used mathematical modeling and/or statistical analysis techniques to introduce variants to a design. Thus, test results anticipate design strategies based on a study of predicted design patterns. Predictive design using these types of mathematical models can be useful to give estimates of these variable indices. However, it may provide a degree of precision that is difficult to quantify and is sometimes inadequate. The quality of the predictive design depends on the type and accuracy of hypotheses, and only those hypotheses, made during modeling. (See generally, U.S. Pat. No. 7,848,908 to Charlier, et al.)

### SUMMARY

Accordingly, one or more of the embodiments presented herein can alter a test micro-environment to, for example, new or optimal shapes that can be incorporated into a predictive design for an article of manufacture. In some embodiments, methods and systems for designing an article of manufacture can use at least one predictor (or set of predictors) that evaluates the expression of some genetic characters of a biological organism, then engineering a phenotypic variation of the organism. In some embodiments, the resultant phenotypic variation can be applied as a predictive design to design and fabricate an article.

According to one embodiment, a method and system is provided to predict a design of an article of manufacture, and can include the steps of: introducing a biological organism into a test environment to activate at least one molecular component of the biological organism representing at least one set of genetic characters of the biological organism; monitoring for change in at least one expression phenomenon pattern in response to the test environment; translating the monitored change in the at least one expression phenomenon pattern into a molecular data profile; interrogating the molecular data profile against a stored database to generate an information pattern of a phenotype correlated to the molecular data profile; and translating the information pattern of the phenotype into a correlated design component of the article of manufacture.

According to one approach, the test environment can include altering at least one of the environmental variables selected from the list consisting of reproductive isolation, resource competition within a organism population, respiration restrictions, surface tension, physical barriers to the growth, pH, temperature, hydrodynamic properties, aerodynamic properties, gravitation pull, atmospheric pressure, atmospheric quality, salinity, UVB, radiation, electromagnetic waves and combinations thereof.

According to one approach, the method and system can also include supplying the translation of the information pattern of the phenotype into a correlated design component of an article; and producing the article by mixing inorganic and organic materials to adapt a composition to a set of physical parameters correlated to at least a macroscopic function of the correlated design component.

In accordance with some embodiments, a computer system is provided to predict a design of an article of manufacture, the computer system comprising: at least one processor; and at least one memory storing executable program instructions. The processor is programmed, via execution of the executable program instructions, to: monitor for a change in at least one expression phenomenon pattern of a biological organism having at least one molecular component activated in a test environment, the biological organism representing at least one set of genetic characters of the biological organism; translate the monitored change in the at least one expression phenomenon pattern into a molecular data profile; interrogate the molecular data profile against a stored database to generate an information pattern of a phenotype correlated to the molecular data profile; and translate the information pattern of the phenotype into a correlated design component of the article of manufacture.

Other features will become more apparent to persons having ordinary skill in the art to which the package pertains and from the following description and claims.

### BRIEF DESCRIPTION OF THE DRAWINGS

The above and other aspects, features and advantages of the present embodiments will be more apparent from the following more particular description thereof, presented in conjunction with the following figures, wherein:

FIG. 1 is a preferred process flow algorithm according to one embodiment of the present methods and systems;

FIG. 2 illustrates a preferred materials processing algorithm of a preferred inorganic-organic hybrid material composition according to one embodiment of the present methods and systems;

FIG. 3 is an algorithm according to an alternate embodiment of the present methods and systems applied to a biological organism;

FIG. 4 is a stress-strain curve for a gradient of mechanical performances for exemplary compositions; and

FIG. 5 illustrates an overview of a preferred predictive design to manufacture process flow algorithm according to one embodiment of the present methods and systems.

FIG. 6 illustrates the system and network environment according to several of the present embodiments; and

FIG. 7 illustrates a simplified block diagram of a processor-based system for implementing methods described according to one or more embodiments.

Corresponding reference characters indicate corresponding components throughout the several views of the drawings.

### DETAILED DESCRIPTION

Despite advances in the art to improve the quality, cost and efficiency of designing products that can operate optimally in their intended environment, improvements are possible and desired. Such improvements could include the use of predictions of at least one specific physical observable trait of an article using a biological model exposed to a predetermined set of environmental variables/stressors, which can be altered compared to found in ambient circumstances. A change of phenotypic expression of an organism as a result of exposure to a predetermined set of environmental stressors can be applied, by analogy, to a change a ‘phenotypic’ change in an article of manufacture that in use can be exposed to those same or similar types of environmental exposures. In some embodiments, methods and systems for designing the article can include a predictive design that can use inorganic and organic materials to fabricate a hybrid article with predicted performance response correlated to predictive information received from results from the predictive design.

Accordingly, one or more of the present embodiments in their most basic form can force nature to create new or optimal shapes that can be incorporated into a predictive design for an article of manufacture. In some embodiments, the present methods and systems for designing an article of manufacture can use at least one predictor (or set of predictors) that evaluates the expression of some genetic characters of a biological organism, then engineering a phenotypic variation of the organism. The resultant phenotypic variation can be applied as a predictive design to design and fabricate an article. In some embodiments, genetic characters can include, for illustrative purposes, characters of a biological organism which can have cells having proteins, enzymes, nucleic acids, lipids, carbohydrates, DNA sequences of any length, RNA sequences of any length, chromosomes, gene assemblies, metabolites, exosomes, circulating micro-organic particles, combinations thereof, and the like.

The following description is not to be taken in a limiting sense, but is made merely for the purpose of describing the general principles of exemplary embodiments. The scope of the embodiments should be determined with reference to the claims. Such predictive designs can be useful in materials processing, consumer products, biomedical applications, and DNA forensics. For illustrative purposes only, one or more of the present embodiments are described to create a predictive design for a recreational surf riding article (surfboard). It is noted though within the scope of some of the present embodiments many other types of articles, such as surfboards, wind boards, snow boards, vehicles (such as cars, boats, submarines, and the like) and be equally applied.

In some embodiments, the organic predictive design can be used to generate many other types of organic, inorganic, and hybrid organic/inorganic designs for articles of manufacture. Examples can include predictive design to create a scaffold to support a bio-molecule or provide forced phenotypic variation of all types of organisms (plant and animal) and even microorganisms. The scaffold can exhibit properties suitable for cementing biological materials, such as calcified connective tissue (e.g., bones). In one embodiment, the article can also be a three dimensional arrangement of individual cells with a morphogenetic derived shape that can allow collection, storage, and release of an organism.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The present predicative design for one or more embodiments for an article of manufacture based on organic models are now described for the development of a surfboard. During the last several decades, very little has been reported in the literature about scientific investigations of the hydrodynamics of surf riding vehicles, such as surfboards. By hydrodynamics, factors such as laminar fluidic flow, turbulent fluidic flow, and a combination thereof can be considered. The same could hold true for snow based vehicles, such as snowboards, or aerodynamic studies for air-craft or air affected craft, such as land and water based vehicles. Further, there are no known predictive design approaches using a bio-inspired design method related to the motion of a surfboard on or in a wave. Ocean wave theory could be applied to explain some of the behavior of a solid object in a propagating wave. Nevertheless, the illustrated predictive design demonstrates how the use of an evolutionary morphogenetic design can predict at least some physical properties, such as hydrodynamics properties and shapes of a surf riding article and/or craft. The present embodiments can even lead to formulating ecological friendly (or “green”) compositions from inorganic and organic nano-materials for the fabrication of the article, thus providing further design optimization.

In one approach, some embodiments can focus to a predictive design method based on the analysis of the morphogenesis of biological organisms, preferably microorganisms such as diatoms, using nucleic acid-based techniques (e.g., gene expression) for the identification and possible prediction of changes in morphology that could be of relevance for shaping a surf board article with improved hydrodynamic properties.

Four classes of biological molecules are known, namely, those having proteins, lipids, carbohydrates and nucleic acids. Nucleic acids, in turn, have two subsumed classes: DNA which is a genetic component of all cells, and RNA which usually functions in a synthesis of proteins.

Some embodiments, for illustrative purposes, are generally described for biomolecules. Nevertheless, for ease of understanding these embodiments, biomolecules having nucleic acids (either DNA or RNA) are described. DNA is emphasized because it is the prime genetic material, carrying all hereditary information within chromosomes. It is noted though that the one or more embodiments are intended to be within the scope of all biomolecules. In this context genetic characters are also referred to a first level of expression of genes or other biological reaction representative of the presence and/or concentration or density of a species or mutation, or deletion of a component of said genetic materials that can result in a change of a second biological response such as phenotypes.

Environmental changes including mechanical forces can also impact nucleic acids integrity. A gene normally exists as two strings of nucleotides entwined in a helical shape resembling a spiral staircase. There are four DNA nucleotides, adenine (A), cytosine (C), guanine (G) and thymine (T), and the sequence of these nucleotides in a gene encodes the information a cell uses to build a specific protein.

Some embodiments are directed towards investigating organisms, including microorganism, such as algae. In one preferred embodiment, the algae can be from the class diatomophyceae (diatoms), especially useful when the test environment involves altered hydrodynamic forces. Diatoms often exhibit relevant hydrodynamic properties in adaptation to their ambient environment. Diatoms are microscopic unicellular aquatic algae found in fresh and salt water. They can have a highly symmetric siliceous cell walls that can adopt various shapes as a result of an evolutionary adaptation to their aquatic environment. Using genome analysis, genetic variability of bio-diversified ocean microorganisms, such as diatoms, can be studied. By controlling the hydrodynamic environment of these microorganisms, their genetic materials (e.g. genes) can produce new metabolites (e.g. proteins) which can also impact the phenotypes (or physical shapes) of such micro-organisms. The present embodiments provide ways to exploit these microorganisms for predicting designs of an inorganic article such as a sport sliding product such as surfboards, snowboards, skateboards, windsurfers, skis, and the like, or any consumer product whose function may require some fluidic or aerodynamic properties for its environmental operation, for example in a fluid or a gas stream, or a hard surface with a low coefficient of friction (e.g., snow or ice).

In one approach, some embodiments can also provide information to assist in the fabrication of the article. This process could, in one embodiment, preferably use a process which could result in a more ecological and material saving production than methods currently used in the art. For example, inorganic-organic hybrid composites are porous materials that can be formed by attaching a layer of organic material on a functionalized surface of an inorganic substrate to form the hybrid composite.

As a specific example, a polysiloxane network can be formed by the condensation of an alkoxy silane, followed by the attachment of the organic polymer layer by the polymerization of monomers to the functionality of the silane surface. Aerogels are among such hybrid composites which are low-density, highly porous nanostructured materials. Crosslinked aerogels, or X-aerogels, are a new class of nanoporous materials made by encapsulating a three-dimensional assembly of silica nanoparticles with polymers. These nano-composite materials are examples of materials that can be processed for the manufacturing of an article created using the predictive design in accordance with some embodiments. It is noted though that a wide variety of inorganic and organic polymer materials can be used and combinations, concentrations, and blend stoichiometry are also ways of practicing this approach. It is noted that the within the scope of some embodiments, a variety of such compositions can considered in addition to the preferred aerogels and cross linked aerogels, such as inorganic composites, inorganic composites, inorganic-organic hybrid composites, nano-composites, porous nano-composites, and the like and combinations thereof.

Thus, one or more embodiments can provide both a predictive design that can include methods for fabricating the resultant design. For the fabrication aspect, manufacturing of a surfboard is not easily amenable to mass manufacturing processes that are also polluting. The present fabrication aspect of some embodiments can provide “green” processing of high performing materials that can not only reduce environmental problems, but also provide higher performing article improved including fluidic and mechanical parameters.

By way of understanding a predictive design for a specific surfboard embodiment, a typical surfboard is about 18-24 inches (46-61 cm) wide, 72-120 inches (183-305 cm) long, and several inches thick. It can have a lightweight, buoyant core covered with a hard shell. In use, the surfer lays face down on the surfboard and paddles out into the ocean to the point where waves begin to rise. The surfer turns the board towards shore, paddles rapidly to match the speed of an incoming wave, then quickly stands up and balances on the board as it is propelled by the face of the breaking wave. One variation of the surfboard is the sail-board, which includes a short mast and sail used for wind surfing. Another variation is the body board, which is shorter than a surf-board and is ridden in the prone position.

Typical Hawaiian surfboards were made of wood from various trees that were carved and shaped by hand, then stained and finished using the natural juices and oils of plants. Large boards up to 144-240 inches (3.6-6 m) long and weight of nearly 200 pounds (91 kg) were fabricated. During the 1920s and 1930s hollow board designs and the use of redwood and balsa laminates reduced the weight.

Many of the first fiberglass surfboard built had two hollow, molded halves with a redwood stiffener, or stringer, running down the center. As this technology eveloved, boards were built with a buoyant, Styrofoam core sandwiched between two thin, plywood veneers and sealed with resin. The birth of the modern surfboard came with the production of boards with polyurethane foam cores. Later, the development of fiberglassing techniques using polyester resins to form the outer shell resulted in the typical contemporary construction of almost every surfboard.

A typical contemporary surfboard can have a rigid polyurethane foam core with an outer shell of fiberglass cloth and polyester resins. If a stringer is used in the design, it is usually made of wood such as redwood, basswood, or spruce. Colored fiberglass stringers can also be used. A fin, or skeg, can be made of wood or laminated layers of fiberglass and resin.

The history of surfboard design shows a passion for constant experimentation. Over the years, most surfboards have been individually designed and hand crafted by talented surfboard builders. Over the last four decades, boards have gotten shorter, then longer, then shorter again. One fin was followed by two fins, then three fins, as builders tried different designs to improve the board's ability to perform maneuvers. Some board builders used channels cut lengthwise along the bottom to improve stability. Today, surfboard builders continue to experiment by trial and error with board design as surfers search for a “perfect board”.

Most surfboards are still built one at a time in small surfboard shops. Although techniques and materials may vary from one surfboard builder to another, the following describes the major steps of the typical process:

(1) Forming the foam core—the foam core, or blank, is formed in a large, cement mold roughly the shape of the surfboard. The mold is constructed in two halves, and the inside is lined with a special paper that keeps the foam from sticking to the mold. The two halves are clamped together and the mold is heated. When the liquid polyurethane chemicals are poured into the mold, the heat triggers a chemical reaction which begins forming dense white foam. Surfboard builders call this process “blowing the blank.” Typically, after 25 minutes, the mold is opened and the foam core is taken out and allowed to finish hardening;

(2) Adding the stringer—Once the core is hard, it can be cut in half vertically from the nose to the tail. A thin stringer is glued between the two halves, and the core is then clamped back together to dry. Stringers provide stiffness and help keep the board from breaking in half;

(3) Shaping the blank—The outline of the finished board can next be traced onto the rough core using a wooden template as a guide. The outline can then be cut out with a saber saw. Starting with the bottom of the blank, the surface is smoothed and contoured to its final shape with a power planer. When the bottom is finished, the board is flipped over and the top is shaped. A power sander removes any ridges left by the planer, and the stringer is contoured with a hand plane. Rough sandpaper is used to shape the sides, or rails. The blank is given a final sanding with fine paper, the position for the fin is marked, and the builder signs the blank with a special design or signature;

(4) Laminating the outer shell—The shaped blank is now ready to be covered with fiberglass and resin to form the hard, outer shell of the surfboard. First, the blank is blown clean with compressed air. If the board is to be colored or have a design on it, acrylic paint is applied directly to the foam with a spray gun or air-brush. When the paint is dry, fiberglass cloth is laid over the surface of the blank and cut to fit. The top of the board, or deck, is laminated first. A polyester resin, known as a laminating resin, is mixed with a second chemical called a catalyst. This starts a chemical reaction which can cause the resin to harden in 15 minutes. The resin is poured over the fiberglass and spread evenly using a rubber squeegee. All of the fiberglass must be covered without leaving too much or too little resin in any spot. This process is known as glassing. When the deck is finished, the board is flipped over and the process repeated on the bottom. The board is then flipped once more, and the deck is given a second layer of fiberglass and resin for added strength and wear resistance. The laminating resin remains slightly tacky and rubbery when dry;

(5) Applying the filler coat and adding the fin—A second coat of resin, called the filler coat or sanding resin, is applied next. The filler coat fills any surface imperfections left in the laminating resin. Sometimes, this coat is called a hot coat resin and contains wax. In either case, this resin contains a slightly different mix of chemicals, which can cause it to harden completely. The deck is coated first and the board is flipped over. The fin is secured with fiberglass tape and a laminating resin. When the fin resin is dry, the bottom of the board and the fin are given a filler coat. When both sides are dry, a small hole is drilled through the tail to attach the leg leash. The leg leash is an elastic cord, sometimes made of surgical rubber tubing that the surfer attaches to one ankle;

(6) Sanding the board—Any excess resin must be carefully sanded away. A power sander is used for the broad surfaces, but the rails and other sharply contoured surfaces are hand sanded to avoid gouging into the fiberglass layer; and

(7) Final finishing—The board is blown clean with compressed air to remove any residual sanding dust. On some boards, decals or color graphics are added at this point. A final coat of gloss resin is then brushed onto the board. Like the other two layers of resin, this final gloss coat is mixed with a catalyst and will harden within 15 minutes. The board is set aside for at least 12 hours to allow the gloss coat to completely harden. As a final step, the board may be wet sanded with very fine sandpaper, then rubbed, buffed, and polished. A surfboard is visually inspected several times during the manufacturing process.

As described, most of the materials and processes used in building a surfboard are hazardous and/or polluting or carcinogenic. The polyurethane chemicals used to make the foam core are toxic and flammable. This process requires explosion-proof fume removal equipment and careful control of the room temperature and humidity. The shaping process produces fine foam dust which can be harmful if inhaled. Finally, the laminating resin gives off poisonous fumes which require the use of an appropriate respirator for the person doing the glassing. The present embodiments can eliminate these material processing deficiencies by providing improved efficiency and cost of manufacturing. Most components using the fabrication aspect of the present embodiments can be eco-friendly. Accordingly, the fabrication process is “green” while the fabricated article can also be eco-friendly when it is recycled or discarded.

Present surfboard design, materials, and construction techniques has produced some improvement through the use of computers—especially those known as computer aided design, or CAD, systems—has simplified the design process. With CAD systems, a board builder can create a three-dimensional picture of a new board design, change dimensions and contours, then print out a finished drawing and contour templates. In the area of materials, some builders have tried boards built with a styrofoam core instead of polyurethane and an epoxy resin instead of polyester. The advantages of this combination are lighter weight, greater strength, and better impact resistance. The epoxy resin also produces less toxic fumes. The disadvantages include greater complexity to the resin preparation process, longer time to manufacture, and significantly greater cost. One variation of this approach can use a graphite fiber cloth for reinforcement rather than glass fiber (fiberglass). This approach adds even more cost and produces boards in only one color—black.

Although surfboard makers have demonstrated ingenuity in design, and CAD systems have assisted in the design outcomes, design attempts and modifications are still largely based on trial and error approach. Some embodiments seek to demonstrate methods and systems that can be implemented improve not only design (here a surfboard), but also fabrication and materials processing. As discussed above, one approach could be the use of Diatoms as an organic predictive design. Diatoms are microscopic single-cell photosynthetic protists (algae) with intricate and cell walls constructed of SiO₂ (glass, formed from silica which is pulled into the cell by transporter proteins). For example, one species, P. tricornutum has been found in several locations around the world, typically in coastal areas with wide fluctuations in salinity. This species can exist in different morphotypes, and changes in cell shape can be stimulated by environmental conditions. This feature can be used to explore the molecular basis of cell shape control and morphogenesis. Furthermore the species can grow in the absence of silicon, and the biogenesis of silicified frustules is facultative, thereby providing opportunities for experimental exploration of hybrid materials and silicon-based nanofabrication in diatoms.

Environmental conditions such as UVB radiation or fluid flow parameters may affect the development of the microorganism or may act as mediating factor affecting various settlement patterns. Early work from Curtis (1961) revealed that cells have rheological properties, namely that it can experience hardening and shape changes on being subject to shear. These changes in shape, mobility and others have been demonstrated to possibly suggest persistent effects on cellular adhesion, cytoskeletal organization, gene expression and others. In one embodiment, the proposed approach describes the exploitation of a confined environment with sub-micron three-dimensional features for settling a microorganism, such as a diatom, in controlled flow regimes (e.g. laminar flow) and identify some genetic disturbances that may result from changes in hydrodynamic forces or other flow derived factors on morphogenetic consequences. These latter can be manipulated to preferably enhance hydrodynamic patterns for genetically building a modified microorganism body shape that could adapt to specific flow patterns, which may involve similar environmental specifications in surfing and/or sliding sport products.

By using computational simulation techniques, one embodiment of an approach could explore diatoms as they could be predicted. A better understanding of diatom adaptation of cell wall structure, and its expression within such a predictive system, help in the simulation and development of better, more realistic evolutionary organisms, particularly in the modeling of realistic biological systems and in the design and generation of both artificial and real entities. Such embodiments can optionally provide combined simulation techniques and nanosystems to model, control and simulate the adaptive processes that govern diatom morphogenesis. Since new walls are formed under cellular control, diatoms offer a model system to study potentially adaptive responses to medium-term environmental pressures.

By confining diatoms into nanofluidic systems, experiments can allow inferred relationships to be isolated and tested. Optionally, computer modeling can be used to further investigate the feasibility of morphological change as an adaptation response to these confinements and environmental stressors. Computer-generated morphological models can be compared directly with the gene expression profiling of the living diatoms. The models can then generate testable genetic modifications to develop predictive morphological changes exploiting the fluid dynamic properties of the environment as a fundamental adaptive mechanism. These changes can occur within the lifetime of a cell and also across generations, allowing the accumulation of new, previously undefined, functional areas in the morphology, and new or improved fluidic motion.

Thus, applied to the specific illustration of a surfboard, some embodiments can also describe the development of hybrid materials (preferably nano-composites) which exhibit improved physico-chemical properties compared to most common materials that are conventionally used in the fabrication of sport products such as surfboards. For example, selected wood species used in the fabrication of surfboards have various values for their bending stresses and modulus elasticity. (See generally, Wood Handbook, Wood as an Engineering Material, US Dept. of Agriculture). Typically, a surfboard is fabricated from two surfaces of fiberglass separated by a foam core. The more a board will flex, the more likely the fiberglass will “buckle”, and lose its strength. Once this occurs, the board begins to break. Thus, the stiffer the board (a result of the use of stiffer woods and higher density core foam), the less likely it is to break. As an example, Western Red Cedar has the lowest combined strength of the woods used by manufacturer of surfboard blanks, such as Clark Foam (and others).

Some embodiments can allow for the production of unprecedented materials properties that could also be tuned and optimized including a gradient of mechanical performances such as from low elastic to high elastic modulus. At the same density (0.5 g/cc) the rupture point (breakage point) of such materials can be over 33 times stronger than polyurethane (PU) foam (e.g. 180 MPa vs 5.5 MPa). For example, when PU foam compresses the material softens. In contrast, X-aerogels actually get stronger when they are compressed.

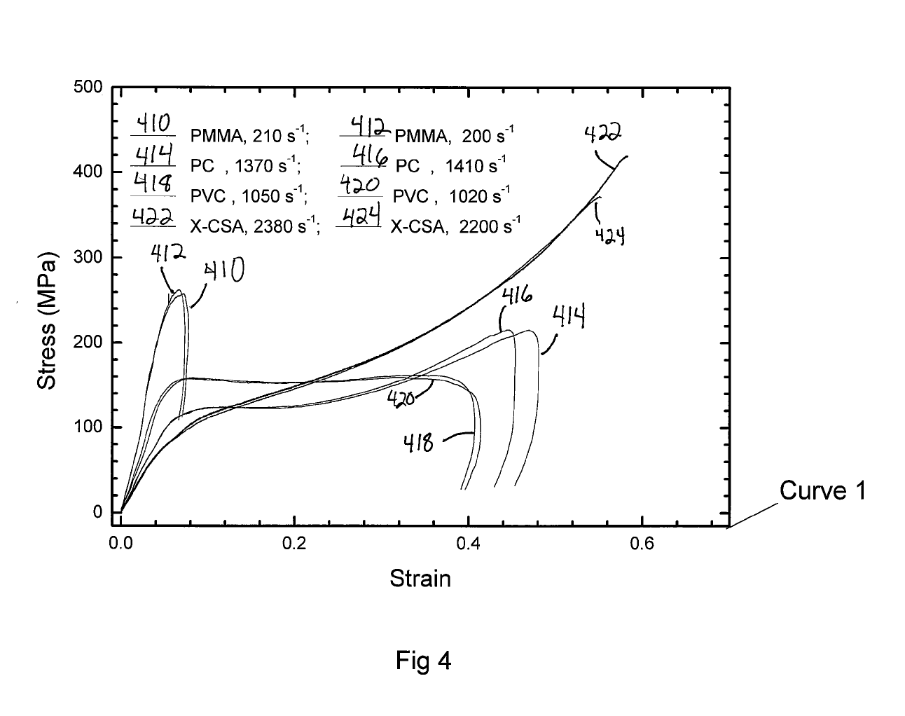

By way of example only and in accordance with one embodiment, FIG. 4 shows a variety of stress-strain curves to illustrate this phenomenon. The examples include compositions for PMMA (Polymethyl methacrylate—a transparent thermoplastic), PC (Polycarbonate), PVC (Polyvinyl Chloride) and X-CSA (cross-linked silica aerogel). As shown, the material is harder when the strain rate is increased. In other words the breakage point is higher (takes more force) when the material is compressed fast than when it's under a pseudo static compression. PU foams such as the ones produced by Clarke Foam usually fail when they are compressed to approximately 40% strain (40% of their original size). In contrast, the proposed nano-composite or X-aerogels can be compressed to over 75% strain before failing. In fact, hybrid materials X-aerogels are capable of absorbing a huge amount of energy (the area under the stress-strain curve).

Accordingly, embodiments utilizing materials such as such as X-CSA and other types of hybrid materials, which can include materials composed of inorganic and organic components, are significantly more environmentally friendly than PU foam. Accordingly, in one embodiment, the composition can be composed of a large portion of nano-engineered Si—O (or sand). In the manufacturing procedure of basic surfboard, the nanomaterials would be hard and smooth making them suitable for glass or carbon fiber lay-ups. The outer surface can also be painted or dyed. These materials can be machined with a broad range of processing techniques such as drilling, molding and so on. Hardness, density, and flexibility of the nanocomposite materials can be controlled allowing a variety of properties for an article, preferably a board product. By adjusting the cross-linking system for a sample at the same density, the material can be engineered to have different mechanical properties.

For example, comparison of various types of other building materials such as polymers and other conventional polyurethane based materials described above, including para-aramid synthetic fiber can be a fiber such as one sold under the trade name KEVLAR-49 epoxy, metals, ceramic materials, and the like versus cross-linked areogels of various formulations show enhanced physical properties that can be tuned accordingly to a set of functions. The cross-linked aerogels have less density than other polymer and/or aluminum based building materials, yet exhibit superior stress, strain and compressive characteristics. For example, the traditional building materials can have specific ultimate compressive strength in the range of about 55,000 to 1,416,000 Nm/Kg compared to various cross-linked aerogel formulations, which can be in the range of about 400,000 to 1,380,000 Nm/Kg. Further, and more surprisingly traditional building materials can have specific energy absorption density in the range of about 4,200 to 35,000 Nm/Kg compared to various cross-linked aerogel formulations, which can be in the range of about 74,000 to 1,350,000 Nm/Kg. Further still, in some embodiments, traditional building materials can have specific energy density G (MPa-m/m) in the range of about 11 to 60 compared to various cross-linked aerogel formulations, which can be about 110.

Thus, in some embodiments, such hybrid materials can include nano-composites such as cross-linked aerogels provide a desirable and unexpected cost effectiveness for the fabrication of a preferred surfboard product. In short, the cost of processing and manufacturing of aerogel based compositions and polymer based compositions are comparable. Although material cost is low for cross-linked aerogels, it can require a relatively expensive supercritical fluid drying step. Nevertheless an aerogel fabrication is preferred because of its reduced negative impact on the environment and superior strain rate and compressive strength. Incorporation of a device and/or sensor for measuring some of the physic-chemical properties of either the article and/or its interaction within its environment is also within the scope of the materials processing of at least some embodiments. For example, strain or pressure sensors as well as biological, chemical or environmental sensing (e.g. bacterial detection, pH, temperature, and the like) could be integrated into the article and outputted for analysis.

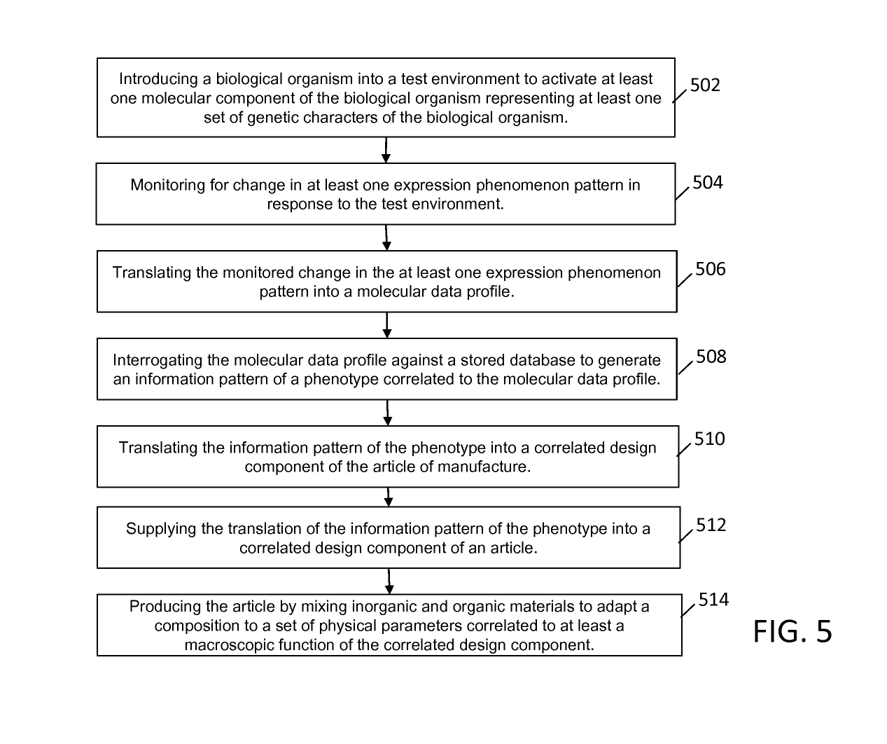

Turning now to the figures, FIG. 5 illustrates an overview of preferred process flow algorithm according to one embodiment of the present methods and systems. In one general embodiment, the process can include methods and systems to predict a design of an article of manufacture, can include the steps of introducing a biological organism into a test environment to activate at least one molecular component of the biological organism representing at least one set of genetic characters of the biological organism at step 502; monitoring for change in at least one expression pattern in response to the test environment at step 504; translating the monitored change in the at least one expression phenomenon pattern into a molecular data profile 506 (e.g., a report); interrogating the molecular data profile against a stored database to generate an information pattern of a phenotype correlated to the molecular profile 508; and translating (e.g., reporting) the information pattern of the phenotype into a correlated design component of an article of manufacture at step 510. Furthermore, in some embodiments, a translation of the information pattern of the phenotype is supplied into a correlated design component of an article at step 512; and the article is produced by mixing inorganic and organic materials to adapt a composition to a set of physical parameters correlated to at least a macroscopic function of the correlated design component in step 514. In a further embodiment, the molecular data profile can include a nucleic acid having a gene and expression pattern obtained from a microarray data analyzed by a bio-informatic algorithm. Algorithms can include statistical and/or mathematical analyses. Elaborations and/or examples of one or more embodiments of the method of FIG. 5 are described in one or more of the method and system embodiments herein.

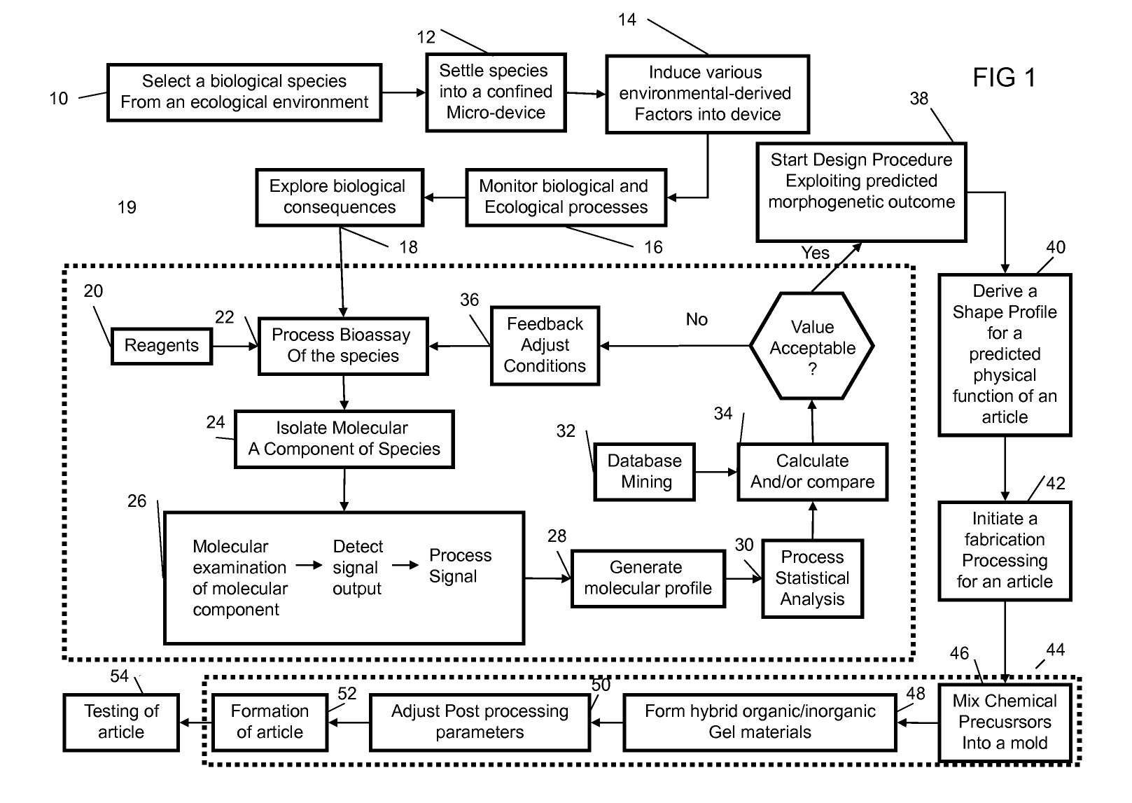

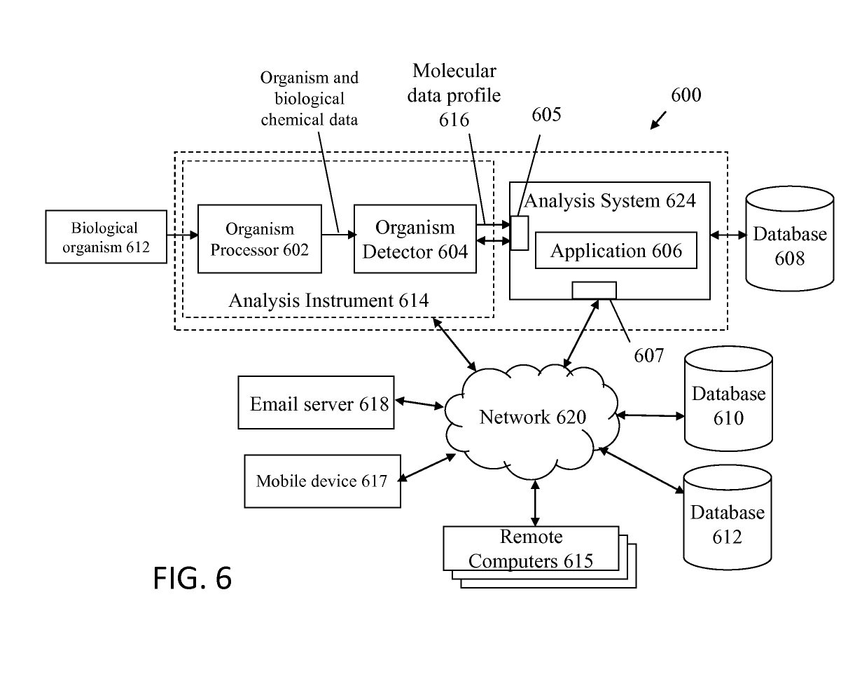

Turning now to FIG. 1, the process can begin at step 10 with selecting a biological species such as a microorganism (e.g. diatom). It is again noted that many types of organisms could be used for this process, but should be able to possess a demonstratable phenotypic change in response to an environmental stressor during its life cycle. FIG. 6 is a schematic diagram of a network environment that can be implemented for one or more of the embodiments described herein, e.g., of FIG. 1 allowing all parts of system of FIG. 1 to be communicatively connected.

Next, at step 12, the species can be placed into a device having a confined and selectively controllable environment. A micro-device can provide controllable physico-chemical properties. Next, at step 14, the system can induce predetermined and controllable environmental factors, which are exposed to the microorganism. Such controllable environmental factors can include: reproductive isolation, resource competition within an organisms population (e.g., malnourishment, sunlight, exposure to toxins, respiration restrictions (e.g., reduced oxygen, surface tension, physical barriers to the growth or development of an organism, variation of pH, temperature, laminar and/or turbulent fluidic flow including drops and/or bubbles (See generally, U.S. Pub. No. 2010/0163412 On-Demand Microfluidic Droplet or Bubble Generation to Attinger et al., and WO 2010/004253 Device and Method of Making Solid Beads to Calder et al.), gravitation pull, atmospheric pressure, aerodynamic properties, salinity, UVB, radiation, any electromagnetic waves that can induce some change of the organic or biological materials, and the like as well as any combination of those and potentially other environmental factors which may impact a phenotypic feature of an exposed organism.

By way of example, at step 18, the system can analyze the gene expression of the genome of the species. For example, if a diatom were grown in a gravity free environment, its changes in morphology could serve as a predictive design for structural design of a space station. The biological species can then monitored at step 16 over a predetermined time interval (e.g. during a life cycle of a diatom). At the end of the predetermined time interval of 16, the system can proceed to step 18 to provide an analysis of any changes in morphology or morphogens. It is noted that any or all of the steps described herein can be manually performed, and/or performed by an automated device, and any combination of automated and manual process steps. The introduction of a physical barrier to development of an organism can be included in some processes of some embodiments, then tested and observed for the expression phenotypic variation. For example, a developmental barrier to vary an expressive phenomenon can be shown in phenotypic changes observed in agricultural products grown in confined barriers. By way of one specific example, in Japan, farmers can grow watermelons or various sizes and shapes by growing the products in glass box receptacles, then allowing them to naturally assume the shape of the receptacle. In this instance, the phenotypic change from a round to a square watermelon is desirable to allow easier stacking and displaying, as well as increase the density of a plurality of products. Using this model, many other types of phenotypic changes of natural organisms can be predicted and tested.

Next, at step 19 the system can provide an analysis of an expression phenomenon such as a gene expression of an exposed microorganism in steps 22 through 36. (See also, Integrated Methods and Systems for Processing a Molecular Profile, PCT/US11/34222; U.S. patent application Ser. No. 13/095842; and U.S. Patent Appl. No. 61/343,337, all of which are incorporated herein by reference in their entirety). Preparation and detection processing can be initiated at step 22. Reagents 20 can be selected to prepare a sample through its processing using at least a bioassay chemistry 22 to preferably extract and/or isolate species of interest at step 24 (e.g. DNA, RNA or proteins). The outcome of an examination sequence within step 26 (namely examination of a molecular component, capture instrument signal output, and process the signal to create a molecular profile can generate a molecular profile or other type of report detailing expressive phenomenon, which can be outputted at step 28. Bioinformatics algorithms and/or other statistical methods at step 30 can be used to calculate and or compare the output from step 28 with comparative molecular profile (or other at step 34 captured by data mining at step 32. For example, public databases such as the Diatom EST database (www.biologic.ens.fr/diatomics/EST/and http://genome.jgi-psf.org/Phatr2/Phatr2.home.html) can be source of genomic information about DNA sequences of a variety of diatoms. The Canadian Diatom database (http://gsc.nrccan.gc.ca/paleo/diatoms/reference_e.php) can also be used as a valuable source of environmental and shape indicators.

In short, the molecular profile is interrogated to possibly quantify any morphogenetic change that may have occurred when exposed to the environmental conditions. If the value generated from this processing step is not acceptable to a threshold value, for example, then a feedback system at step 36 can adjust the bioassay conditions to process the species (e.g. increase in DNA concentrations). If the value is acceptable then the system can output a morphogenetic outcome at step 38.

From the morphogenic outcome in step 38, the system can, as generally shown in FIG. 5, it a general form, at step 512 manufacture a design by receiving a supplied translation of the information pattern of the phenotype into a correlated design component of an article from step 38, then at step 514 produce the article by mixing inorganic and organic materials to adapt a composition to a set of physical parameters correlated to at least a macroscopic function of the correlated design component. Thus, as shown more specifically in FIG. 1, from the morphogenic outcome in step 38, a shape profile (or other type of expressive phenomenon profile) adapted to the controlled properties of the environment, which can be exhibited from a change in preferably some physical properties (e.g., elasticity) in step 40. The profile of step 40 can be analyzed for a predictive design of an article such as a surfboard. Next, at step 42, article fabrication can be initiated using the predictive design through preferably a hybrid chemical and materials processing scheme 44. It is noted that other types of expressive phenomenon profiles can be provided within the scope of at least some embodiments of the present invention. By way of an alternate example, silica uptake of a diatom colony exposed to a turbulent fluid could also be recorded and outputted from the system and compared to other databases for silica uptake. In fact in a constrained environments other metabolites, proteins, or anything that an organism released and a device can quantify can be tested can also be considered within the scope of the present environments. Further more than one of these characteristics can be captured and analyzed.

Materials processing algorithm 44 can provide for an organic and inorganic composite components that comprises mixing chemical components at step 46, forming gels at step 48, adjusting post processing conditions at step 50 to desired specifications for forming an article at step 52. Optionally, the article can be tested, which might employ the application of sensors to measure various design parameters that can undergo properties and/or performance testing at step 54. It is noted that one or more, including all, of the processing steps can be automated. Accordingly, the article is “predictively shaped” (i.e. designed) for specific flow regimes while optimizing the materials composition to adapt the surf product in light of flow derived factors and hydrodynamic forces that may be encounter at a macro-scale in sea conditions. The fabrication of said article is preferably processed by using some hybrid nano-composites whose properties can be tuned to some targeted functions and properties to mass produce and duplicate the article through eco-friendly processing.

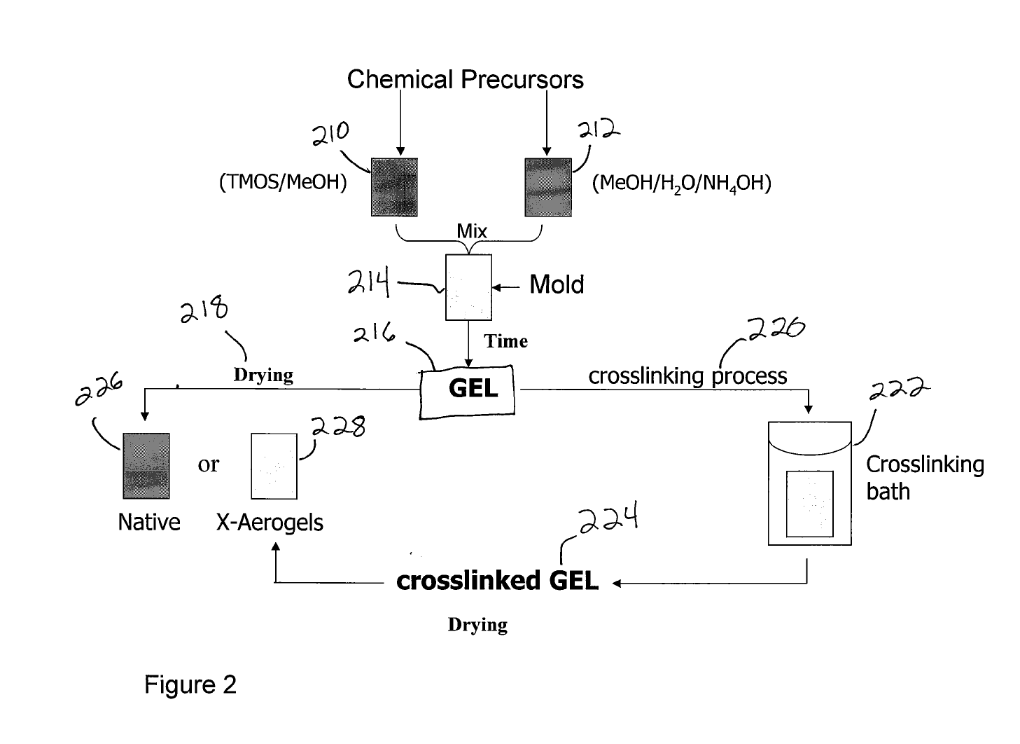

Turning now to FIG. 2, there is illustrated a materials processing algorithm (i.e., a more detailed algorithm of step 44 FIG. 1). The processing of nanocomposite materials can comprises the mixture of chemical precursors 210 and 212 (e.g. TMOS/MeOH or MeOH/H2O/NH4OH) into a mold 214 to subsequently form gel materials 216. Gel 216 can then be dried at 218 (e.g. using super-critical processing) to form a native hybrid inorganic-organic gel 226. Alternately, gel 216 can undergo an additional cross-linking process 220 through a cross-linking bath 222 that can form a cross-linked gel 224, which is then dried to form a hybrid inorganic-organic X-aerogels.

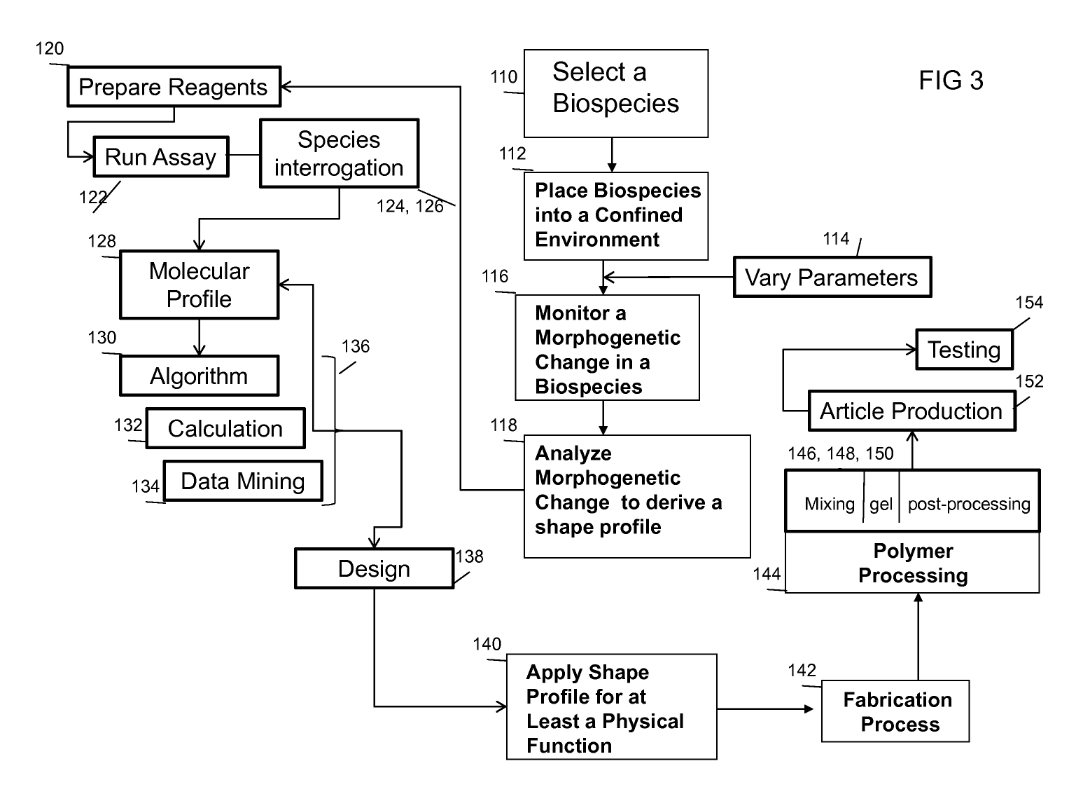

FIG. 3 illustrates an alternate approach in accordance with other embodiments. As shown, a biological species (organism) is selected from a specific ecological environment (e.g. diatom in ocean) at step 110 and placed into, for example a confined three-dimensional microdevice such as a lab-on-chip system 112 where various environmental parameters, as described herein, 114 can be varied. For example, the assembly of nanoscale to macro scale features of this microdevice could provide some fluid flow gradient from laminar to turbulent. These parameters could be tuned and manipulated in controlled cycle times or spatially within the microdevice to induce some phenotypic changes onto the species which can be monitored at step 116.

Monitoring morphogenic change in a bio-species at step 116 can include exploring some biological consequences that could be manifested by changes in physical traits and/or genetic variations, which can be analyzed at step 118. To identify such changes, some bioassays can be performed for profiling some molecular information. Reagents can be prepared at step 120 for processing some predetermined bioassay reaction 122, which can result in the isolation of some molecular component of the species 124 such as nucleic acid molecules (e.g. DNA, RNA) or proteins, sugars and other molecular components. A process step 126 of examination of at least one of these molecular components can generate a signal output (e.g. optical emission such as Raman, Fluorescence) which can be processed for analysis, for example by comparison with a reference signal data or set of data, preferably using some statistical method (e.g. principal component analysis, neural networking, fuzzy logic). The analysis can result in establishing a molecular profile at step 128.

The molecular profile from step 128 can then be interrogated by statistical algorithms at step 130 for initiating a mathematical processing which may preferably comprise calculations 132, data mining 134 that can generate either a feedback processing 136 for improving some quality factors (e.g. accuracy, robustness) of the overall processing scheme or providing a set of information amenable to starting a design procedure based upon various outcomes that can comprise morphogenetic outcome.

The result analysis of the molecular profile can be initiate a design procedure at step 138, which can then derive an article shape profile 140 for at least one predicted physical function of relevance for performances, aesthetics, robustness, quality or other factors of value for a functional article such as sport product, including surfboards, skateboards, ski, or other sliding article.

Based on the design of the article, a fabrication process can be initiated at step 142, which may preferably comprise using polymer processing at step 144 (see e.g., FIG. 3). A preferred polymer processing sequence can include the steps of mixing chemical precursors at step 146 to form a hybrid gel materials 148 (e.g. sol-gel, nano-composite, co-polymers) which can then be post-processed by different step and parameters 150 to form an article at step 152 compatible with mass manufacturing techniques (e.g. molding). A next step and optional step 154 of quality control and testing of the article can then be initiated for validating the predicted performances of the article, for example hydrodynamic performances for a surfboard or size and/or shape of a cell from a scaffold substrate used for collecting and/or processing a bio-specimen for its storage or processing using some assay chemistry to extract a biomolecule of interest such as nucleic acids.

For ease in understanding the connectivity and functionality of some embodiments of the system, FIG. 6 illustrates a system 600 according to several embodiments. As shown, system 600 includes an analysis instrument 614 comprising an organism processor 602 to provide a confined environment that is alterable for testing and a detector 604 to measure the at least one expression pattern of the organism in response to the altered environment as part of an analysis system 624 executing an analysis application 606, and a local database 608. The system 600 can also includes remote databases 610 and 612, remote computers 616, mobile device 617 and an email server 618 variously coupled to the analysis instrument 614 and/or the analysis system 624 via a network 620. Network 620 may be one or more of any wired and/or wireless point-to-point connection, local area network, wide area network, internet, intranet and so on.

In one embodiment, the organism processor 602 of the analysis instrument 614 receives an organism. The organism processor may include one or more sample processing techniques such as those described herein to prepare the altered micro-environment for detection or interrogation by the organism detector 604. In one embodiment, the organism processor can output the organism and any biological and/or chemical data pertaining to the organism to the organism detector 604. The organism detector 604 can detect or interrogates the organism or sample using one or more detection devices such as those described herein.

In one embodiment, the organism detector 604 outputs a molecular data profile 616 to an input or interface 605 of the analysis system 624 and in another embodiment, the molecular data profile 616 is output to the analysis system 624 via the network 620 and an interface 607 of the analysis system 624. It is understood that the interfaces 605 and 607 may be any wireless, wired or optical input that can interface with the corresponding device. For example, interface 607 may be a network interface. It is understood that the analysis instrument 614 and the analysis system 624 (e.g., the interfaces 605 and 607) can include the appropriate wireless, wired, and/or optical transmitting and receiving devices to effect the transfer of signaling therebetween and with the network 620 and any databases. In some embodiments, the analysis instrument 614 may be an automated and integrated device having both the organism processor 602 and the organism detector 604. In such integrated embodiments, the organism may be moved within the instrument 614 in a manner that is automated and not susceptible to contamination through handling. In other embodiments, the organism processor 602 and the organism detector 604 are not integrated and these functions may be performed by separate devices that may or may not be communicationally coupled together. For example, in one embodiment, the data output of the organism processor 602 is electronically transmitted or delivered to the organism detector 604 (directly via interface 605 or indirectly via the network 620 and interface 607), while the prepared organism can physically transported (e.g., within a carrier or cartridge) to the organism detector 604. In another embodiment, the data output of the organism processor 602 is directly electronically transmitted or delivered to the detector 604 (or processor controlling the specimen detector), while the prepared organism is physically transported (e.g., within a carrier or cartridge) within the analysis instrument 614 to the portion functioning as the detector 604. In some embodiments, the analysis instrument 614 is a processor-based device having at least one microprocessor, at least one memory device or computer readable medium, and executable program instructions stored by the at least one memory device or computer readable medium and executed by the at least one microprocessor to perform one or more functions, such as those described herein. Additionally, the hardware and software (or firmware) control components of the analysis instrument provide for the control of any included devices that process or detect the specimen.

In one embodiment, the analysis system 624 has stored thereon or stored in a memory or computer readable medium accessible by the analysis system software for executing the analysis application 606 for receiving and processing information and data from the analysis instrument 614, such as a molecular profile 616, and generating various outputs, such as report. In one embodiment, the application 606 further comprises one or more functional application components for processing the input from the analysis instrument 614, data mining, data comparisons, report generation, quality control checks, result reporting and/or transmitting adjustments back to one or both of the organism processor 602 and the organism detector 604. The output of the organism detector 604 is provided at the input or the interface 605 of the analysis system 624. The interface may be a physical input, output or connector, or may be a functional interface implemented in received signaling in the control signaling or processor of the analysis system 624. The interface 605 may be a direct input from the organism detector 604 (as illustrated) or may be the interface 607, e.g., a network interface that receives data or electronic signaling representing the molecular profile from the network 620.

In some embodiments, the organism processor 602 and/or the organism detector 604 are proximate to the analysis system 624. In other embodiments, the organism processor 602 and/or the organism detector 604 are remotely located from the analysis system 624, e.g., the organism processor 602 and/or the organism detector 604 are integrated into a field device that is local to the specimen/organism collection location and the analysis system 624 is remotely located at a laboratory or other analysis location. In such case, the output of the organism processor 602 and/or the organism detector 604 is automatically and electronically delivered to the analysis system 624, e.g., using the network 620. This received data may be stored at the analysis system 624 or a local database 608 coupled thereto. In some embodiments, output from the analysis instrument 614 is stored at a remote computer 615 and/or a remote database 610 and/or 612, such that the information or output can be accessed by or retrieved by the analysis system 624 automatically or responsive to a user's instructions at the analysis system 624.

In one embodiment, the analysis system 624 comprises a computer or computer device including at least one processor, at least one memory or computer readable medium that stores and executes computer program code to implement its functionality. In one embodiment, the computer program code is remotely stored and executed for example, using a remote server or remote computer 615, and the analysis system 624 serves as the user interface. In one embodiment, the analysis system 624 is a single computer or computer device, whereas in other embodiments, the analysis system 624 is a plurality of computers or computer devices working together to implement the functionality of the analysis system.

In one embodiment, the computer or computer devices of the analysis system 624, the remote computers 615, and/or the mobile device 617 can be personal computers in communication with one or more other devices via the network 620 (i.e., any network accessible, enabled or addressable device, for example, in the case of the internet, a device having an IP address). Example computers or computer devices include but are not limited to desktop computers, laptop computers, personal data assistants (PDAs), smartphones, touch screen computing devices, handheld computing devices, or any other computing device having functionality to couple to the network 620. Additionally, the application 606 may also include a web application that acts to serve web content to other remote computers (e.g., remote computers 614, mobile device 617, etc.). These remote computes may include web browser capabilities and are able to access the web application component of the application 606 using a web browser to interact with and/or control one or more automated processes of the analysis system 64 and/or the analysis instrument 614.

In one or more embodiments, the system 600 includes remote databases 610 and 612 that may be accessed by the analysis system 624 or written to by the analysis system 624 and/or the analysis instrument 614. In some embodiments, databases 610 and 612 can be used to store predetermined databases for comparisons or correlations with data relating to collected specimens processed by the analysis instrument 614, such as the generated molecular data profile 616.

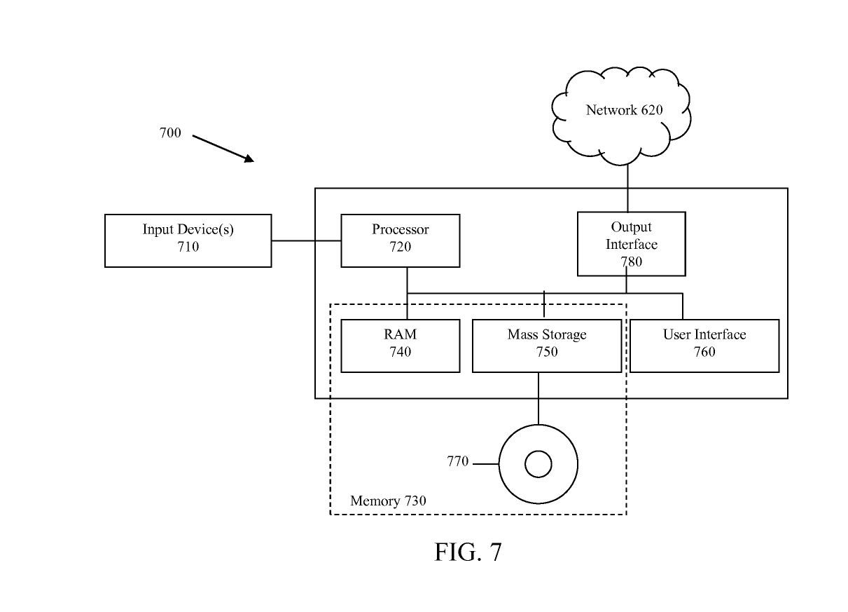

The methods and processes described herein may be utilized, implemented and/or run on many different types of systems. Referring to FIG. 7, there is illustrated a processor-based system 700 that may be used for any such implementations. One or more components of the system 700 may be used for implementing one or more of the control components of one or more of the analysis instrument 614, the analysis system 624, the remote computers 615 and the remote device 617. By way of example, the system may comprise at least one processor 720 (also referred to as a Central Processing Unit (CPU); a memory 730 (also referred to as a computer readable medium) generally including a Random Access Memory (RAM) 740, a mass storage unit 750 (such as a disk drive), and an external memory 770; and a user interface 760 such as a display. The processor 720 may be used to execute or assist in executing the steps of the methods and techniques described herein. The system may further comprise one or more input devices 710. The devices may comprise any user input device such a keyboard, mouse, touch screen keypad or keyboard. The input devices 710 may further comprise one or more processing and/or detection hardware components.

The mass storage unit 750 may also be referred as a memory and may include or comprise any type of computer readable storage or recording medium or media. The computer readable storage or recording medium or media may be fixed in the mass storage unit, or the mass storage unit may optionally include an external memory device 770, such as a digital video disk (DVD), Blu-ray disc, compact disk (CD), USB storage device, floppy disk, RAID disk drive or other media. By way of example, the mass storage unit 470 may comprise a disk drive, a hard disk drive, flash memory device, USB storage device, Blu-ray disc drive, DVD drive, CD drive, floppy disk drive, RAID disk drive, etc. The mass storage unit 750 or external memory device 770 may be used for storing executable program instructions or code that when executed by the processor, implements the methods and techniques described herein. Any of the applications and/or components described herein may be expressed as a set of executable program instructions that when executed by a processor (such as CPU), can performed one or more of the functions described in the various embodiments herein. It is understood that such executable program instructions may take the form of machine executable software or firmware, for example, that may interact with one or more hardware components or other software or firmware components.

Thus, external memory device 770 may optionally be used with the mass storage unit 750, and may be collectively referred to as the memory 730, which may be used for storing code that implements the methods and techniques described herein. However, any of the storage devices, such as the RAM or mass storage unit, may be used for storing such code. For example, any of such storage devices may serve as a tangible computer storage medium for embodying a computer program for causing a computer or display device to perform the steps of any of the methods, code, and/or techniques described herein. Furthermore, any of the storage devices, such as the RAM 740 or mass storage unit 750, may be used for storing any needed database(s). Furthermore, the system 700 may include external outputs at an output interface 780 to allow the system to output data or other information to other servers, network components or computing devices in the overall system 600 via one or more networks 620, such as described throughout this application.

While the figures and descriptions herein have been described in conjunction with specific embodiments, many alternatives, modifications, and variations will be apparent to those skilled in the art in light of the foregoing description. Changes in form, as well as substitution of equivalents, are contemplated as circumstances may suggest or render expedient.

1. A method to predict a design of an article of manufacture, comprising the steps of:
introducing a biological organism into a test environment to activate at least one molecular component of the biological organism representing at least one set of genetic characters of the biological organism;
monitoring for a change in at least one expression phenomenon pattern in response to the test environment;
translating the monitored change in the at least one expression phenomenon pattern into a molecular data profile;
interrogating the molecular data profile against a stored database to generate an information pattern of a phenotype correlated to the molecular data profile; and
translating the information pattern of the phenotype into a correlated design component of the article of manufacture.

2. The method of claim 1, wherein the test environment can include altering at least one of the environmental variables selected from the list consisting of reproductive isolation, resource competition within a organism population, respiration restrictions, surface tension, physical barriers to the growth, pH, temperature, hydrodynamic properties, aerodynamic properties, gravitation pull, atmospheric pressure, atmospheric quality, salinity, UVB, radiation, electromagnetic waves and combinations thereof.

3. The method of claim 2, wherein hydrodynamic properties are selected from the list consisting of laminar fluidic flow, turbulent fluidic flow, and a combination thereof.

4. The method of claim 1, wherein the test environment for correlated design component comprises hydrodynamic forces.

5. The method according to claim 1, wherein the biological organism that is an algae.

6. The method of claim 5, wherein the algae is from the class Diatomophyceae, and the test environment comprises altered hydrodynamic forces.

7. The method according to claim 1, wherein the molecular data profile comprises a nucleic acid having a gene and expression pattern obtained from a microarray data analyzed by a bio-informatic algorithm.

8. The method according to claim 4, wherein the article is a sport sliding product selected from the list consisting of surfboards, snowboards, skateboards, windsurfers, and skis.

9. The method according to claim 1, wherein the organism can comprise cells having components selected from the list consisting of proteins, enzymes, nucleic acids, lipids, carbohydrates, DNA sequences of any length, RNA sequences of any length, chromosomes, gene assemblies, metabolites, exosomes, circulating micro-organic particles, and combinations thereof.

10. The method according to claim 9, wherein the three dimensional arrangement can form a scaffold.

11. The method according to claim 10 wherein the scaffold exhibits properties suitable for cementing biological materials.

12. The method of claim 11, wherein the biological material is calcified connective tissue.

13. The method of claim 1, wherein the genetic characters of the biological organism can be selected from the list consisting of: proteins, enzymes, nucleic acids, lipids, carbohydrates DNA sequences of any length, RNA sequences of any length, chromosomes, gene assemblies, metabolites, exosomes, circulating micro-organic particles, and combinations thereof.

14. A method to manufacture the design of claim 1, comprising the steps of:
supplying the translation of the information pattern of the phenotype into a correlated design component of an article; and
producing the article by mixing inorganic and organic materials to adapt a composition to a set of physical parameters correlated to at least a macroscopic function of the correlated design component.

15. The method according to claim 14 wherein the materials are selected from the list consisting of cross-linked aerogels, nano-composites, hybrid inorganic-organic materials, and combinations thereof.

16. The method according to claim 14, wherein the article is a three dimensional arrangement of individual cells with a morphogenetic derived shape that can allow collection, storage, and release of an organism.

17. A computer system to predict a design of an article of manufacture, the computer system comprising:
at least one processor; and
at least one memory storing executable program instructions,
wherein the processor is programmed, via execution of the executable program instructions, to:
monitor for a change in at least one expression phenomenon pattern of a biological organism having at least one molecular component activated in a test environment, the biological organism representing at least one set of genetic characters of the biological organism;
translate the monitored change in the at least one expression phenomenon pattern into a molecular data profile;
interrogate the molecular data profile against a stored database to generate an information pattern of a phenotype correlated to the molecular data profile; and
translate the information pattern of the phenotype into a correlated design component of the article of manufacture.
